(19) United States
(12) Patent Application Publication
(10) Pub. No.: US 2026/0108177 A1
(43) Pub. Date: Apr. 23, 2026

(54) SYSTEM AND METHOD FOR DETECTING AND ACCOMODATING A GAIT OF A USER

(71) Applicant: GM Global Technology Operations LLC, Detroit, MI (US)

(72) Inventors: Mitesh Lalwala, Sterling Heights, MI (US); Siddhartha Gupta, Rochester Hills, MI (US); Senthil Karuppaswamy, Rochester Hills, MI (US); Alok Warey, Novi, MI (US)

(73) Assignee: GM Global Technology Operations LLC, Detroit, MI (US)

(21) Appl. No.: 18/922,771

(22) Filed: Oct. 22, 2024

(51) Int. Cl. A61B 5/11

(52) U.S. Cl. CPC A61B 5/112; A61B 2562/02

(57) ABSTRACT

A computer-implemented method that, when executed by data processing hardware, causes the data processing hardware to perform operations comprising detecting one or more individuals within a proximity of a vehicle, gathering data concerning the one or more individuals, calculating a baseline gait for each of the one or more individuals, determining a physiology for each of the one or more individuals based on the data and the baseline gait, and calculating one or more vehicle settings based on the physiology and the baseline gait for each of the one or more individuals.

### INTRODUCTION

The information provided in this section is for the purpose of generally presenting the context of the disclosure. Work of the presently named inventors, to the extent it is described in this section, as well as aspects of the description that may not otherwise qualify as prior art at the time of filing, are neither expressly nor impliedly admitted as prior art against the present disclosure.

The present disclosure relates generally to vehicles and, more particularly, to a system and method of evaluating one or more individuals and adjusting a vehicle accordingly.

Vehicles commonly include one or more settings and features that can be manually adjusted by the users of the vehicle. From time to time, the settings or features need to be modified and this requires the one or more users to manually adjust the settings or features. Shortcomings of existing systems and methods are addressed by one or more aspects of the present disclosure.

### SUMMARY

In one configuration, a computer-implemented method that, when executed by data processing hardware, causes the data processing hardware to perform operations is provided. The operations include detecting one or more individuals within a proximity of a vehicle, gathering data concerning the one or more individuals, calculating a baseline gait for each of the one or more individuals, determining a physiology for each of the one or more individuals based on the data and the baseline gait, and calculating one or more vehicle settings based on the physiology and the baseline gait for each of the one or more individuals.

The method may include one or more of the following optional aspects or steps. For example, the method further includes determining whether the data indicates one or more deviations from the baseline gait and modifying the vehicle settings based on the one or more deviations.

According to another aspect, detecting the one or more individuals within the proximity of the vehicle further includes detecting a key fob or a mobile device.

According to at least one example, gathering the data concerning the one or more individuals further includes capturing the data using one or more sensors coupled to the vehicle.

According to another example, the method further includes identifying the one or more individuals using facial recognition.

According to at least one aspect, calculating the one or more vehicle settings based on the physiology and the baseline gait for each of the one or more individuals further includes calculating a position of one or more seats arranged within the vehicle.

According to another aspect, calculating the one or more vehicle settings based on the physiology and the baseline gait for each of the one or more individuals further includes calculating a position of a steering wheel arranged within the vehicle.

According to at least one example, calculating the one or more vehicle settings based on the physiology and the baseline gait for each of the one or more individuals further includes opening a liftgate of the vehicle.

According to another example, calculating the one or more vehicle settings based on the physiology and the baseline gait for each of the one or more individuals further includes calculating a position of one or more sideview mirrors.

According to at least one aspect, calculating the one or more vehicle settings based on the physiology and the baseline gait for each of the one or more individuals further includes calculating a position of a rearview mirror.

In another configuration, a system is provided and includes data processing hardware and memory hardware in communication with the data processing hardware, the memory hardware storing instructions that, when executed on the data processing hardware, cause the data processing hardware to perform operations. The operations include detecting one or more individuals within a proximity of a vehicle, gathering data concerning the one or more individuals, calculating a baseline gait for each of the one or more individuals, determining a physiology for each of the one or more individuals based on the data and the baseline gait, and calculating one or more vehicle settings based on the physiology and the baseline gait for each of the one or more individuals.

The system may include one or more of the following optional aspects or steps. For example, the system further includes determining whether the data indicates one or more deviations from the baseline gait.

According to at least one aspect, detecting the one or more individuals within the proximity of the vehicle further includes detecting a key fob or a mobile device.

According to another aspect, gathering the data concerning the one or more individuals further includes capturing the data using one or more sensors coupled to the vehicle.

According to at least one example, the system further includes identifying the one or more individuals using facial recognition.

In another configuration, a vehicle management system is provided and includes a sensor system including one or more sensors coupled to a vehicle, data processing hardware, and memory hardware in communication with the data processing hardware, the memory hardware storing instructions that, when executed on the data processing hardware, cause the data processing hardware to perform operations. The operations include detecting one or more individuals within a proximity of the vehicle, gathering data concerning the one or more individuals, calculating a baseline gait for each of the one or more individuals, determining a physiology for each of the one or more individuals based on the data and the baseline gait, and calculating one or more vehicle settings based on the physiology and the baseline gait for each of the one or more individuals.

The vehicle management system may include one or more of the following optional aspects or steps. For example, the vehicle management system further includes determining whether the data indicates one or more deviations from the baseline gait.

According to at least one aspect, detecting the one or more individuals within the proximity of the vehicle further includes detecting a key fob or a mobile device.

According to another aspect, gathering the data concerning the one or more individuals further includes capturing the data using the one or more sensors.

According to at least one example, the vehicle management system further includes identifying the one or more individuals using facial recognition.

### BRIEF DESCRIPTION OF THE DRAWINGS

The drawings described herein are for illustrative purposes only of selected configurations and are not intended to limit the scope of the present disclosure.

FIG. 1 is a schematic view of a vehicle operating environment including a vehicle and a vehicle management system according to principles of the present disclosure;

FIG. 2 is a top view of the vehicle of FIG. 1 according to the principles of the present disclosure;

FIG. 3 is a fragmentary perspective view of an interior of the vehicle of FIG. 1;

FIG. 4 is a schematic view of the vehicle management system of FIG. 1; and

FIG. 5 is a flow diagram of a method of evaluating gait of one or more individuals and calculating one or more vehicle settings according to the principles of the present disclosure.

Corresponding reference numerals indicate corresponding parts throughout the drawings.

### DETAILED DESCRIPTION

Example configurations will now be described more fully with reference to the accompanying drawings. Example configurations are provided so that this disclosure will be thorough, and will fully convey the scope of the disclosure to those of ordinary skill in the art. Specific details are set forth such as examples of specific components, devices, and methods, to provide a thorough understanding of configurations of the present disclosure. It will be apparent to those of ordinary skill in the art that specific details need not be employed, that example configurations may be embodied in many different forms, and that the specific details and the example configurations should not be construed to limit the scope of the disclosure.

The terminology used herein is for the purpose of describing particular exemplary configurations only and is not intended to be limiting. As used herein, the singular articles “a,” “an,” and “the” may be intended to include the plural forms as well, unless the context clearly indicates otherwise. The terms “comprises,” “comprising,” “including,” and “having,” are inclusive and therefore specify the presence of features, steps, operations, elements, and/or components, but do not preclude the presence or addition of one or more other features, steps, operations, elements, components, and/or groups thereof. The method steps, processes, and operations described herein are not to be construed as necessarily requiring their performance in the particular order discussed or illustrated, unless specifically identified as an order of performance. Additional or alternative steps may be employed.

When an element or layer is referred to as being “on,” “engaged to,” “connected to,” “attached to,” or “coupled to” another element or layer, it may be directly on, engaged, connected, attached, or coupled to the other element or layer, or intervening elements or layers may be present. In contrast, when an element is referred to as being “directly on,” “directly engaged to,” “directly connected to,” “directly attached to,” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

The terms “first,” “second,” “third,” etc. may be used herein to describe various elements, components, regions, layers and/or sections. These elements, components, regions, layers and/or sections should not be limited by these terms. These terms may be only used to distinguish one element, component, region, layer or section from another region, layer or section. Terms such as “first,” “second,” and other numerical terms do not imply a sequence or order unless clearly indicated by the context. Thus, a first element, component, region, layer or section discussed below could be termed a second element, component, region, layer or section without departing from the teachings of the example configurations.

In this application, including the definitions below, the term “module” may be replaced with the term “circuit.” The term “module” may refer to, be part of, or include an Application Specific Integrated Circuit (ASIC); a digital, analog, or mixed analog/digital discrete circuit; a digital, analog, or mixed analog/digital integrated circuit; a combinational logic circuit; a field programmable gate array (FPGA); a processor (shared, dedicated, or group) that executes code; memory (shared, dedicated, or group) that stores code executed by a processor; other suitable hardware components that provide the described functionality; or a combination of some or all of the above, such as in a system-on-chip.

The term “code,” as used above, may include software, firmware, and/or microcode, and may refer to programs, routines, functions, classes, and/or objects. The term “shared processor” encompasses a single processor that executes some or all code from multiple modules. The term “group processor” encompasses a processor that, in combination with additional processors, executes some or all code from one or more modules. The term “shared memory” encompasses a single memory that stores some or all code from multiple modules. The term “group memory” encompasses a memory that, in combination with additional memories, stores some or all code from one or more modules. The term “memory” may be a subset of the term “computer-readable medium.” The term “computer-readable medium” does not encompass transitory electrical and electromagnetic signals propagating through a medium, and may therefore be considered tangible and non-transitory memory. Non-limiting examples of a non-transitory memory include a tangible computer readable medium including a nonvolatile memory, magnetic storage, and optical storage.

The apparatuses and methods described in this application may be partially or fully implemented by one or more computer programs executed by one or more processors. The computer programs include processor-executable instructions that are stored on at least one non-transitory tangible computer readable medium. The computer programs may also include and/or rely on stored data.

A software application (i.e., a software resource) may refer to computer software that causes a computing device to perform a task. In some examples, a software application may be referred to as an “application,” an “app,” or a “program.” Example applications include, but are not limited to, system diagnostic applications, system management applications, system maintenance applications, word processing applications, spreadsheet applications, messaging applications, media streaming applications, social networking applications, and gaming applications.

The non-transitory memory may be physical devices used to store programs (e.g., sequences of instructions) or data (e.g., program state information) on a temporary or permanent basis for use by a computing device. The non-transitory memory may be volatile and/or non-volatile addressable semiconductor memory. Examples of non-volatile memory include, but are not limited to, flash memory and read-only memory (ROM)/programmable read-only memory (PROM)/erasable programmable read-only memory (EPROM)/electronically erasable programmable read-only memory (EEPROM) (e.g., typically used for firmware, such as boot programs). Examples of volatile memory include, but are not limited to, random access memory (RAM), dynamic random access memory (DRAM), static random access memory (SRAM), phase change memory (PCM) as well as disks or tapes.

These computer programs (also known as programs, software, software applications or code) include machine instructions for a programmable processor, and can be implemented in a high-level procedural and/or object-oriented programming language, and/or in assembly/machine language. As used herein, the terms “machine-readable medium” and “computer-readable medium” refer to any computer program product, non-transitory computer readable medium, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The term “machine-readable signal” refers to any signal used to provide machine instructions and/or data to a programmable processor.

Various implementations of the systems and techniques described herein can be realized in digital electronic and/or optical circuitry, integrated circuitry, specially designed ASICs (application specific integrated circuits), computer hardware, firmware, software, and/or combinations thereof. These various implementations can include implementation in one or more computer programs that are executable and/or interpretable on a programmable system including at least one programmable processor, which may be special or general purpose, coupled to receive data and instructions from, and to transmit data and instructions to, a storage system, at least one input device, and at least one output device.

The processes and logic flows described in this specification can be performed by one or more programmable processors, also referred to as data processing hardware, executing one or more computer programs to perform functions by operating on input data and generating output. The processes and logic flows can also be performed by special purpose logic circuitry, e.g., an FPGA (field programmable gate array) or an ASIC (application specific integrated circuit). Processors suitable for the execution of a computer program include, by way of example, both general and special purpose microprocessors, and any one or more processors of any kind of digital computer. Generally, a processor will receive instructions and data from a read only memory or a random access memory or both. The essential elements of a computer are a processor for performing instructions and one or more memory devices for storing instructions and data. Generally, a computer will also include, or be operatively coupled to receive data from or transfer data to, or both, one or more mass storage devices for storing data, e.g., magnetic, magneto optical disks, or optical disks. However, a computer need not have such devices. Computer readable media suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto optical disks; and CD ROM and DVD-ROM disks. The processor and the memory can be supplemented by, or incorporated in, special purpose logic circuitry.

To provide for interaction with a user, one or more aspects of the disclosure can be implemented on a computer having a display device, e.g., a CRT (cathode ray tube), LCD (liquid crystal display) monitor, or touch screen for displaying information to the user and optionally a keyboard and a pointing device, e.g., a mouse or a trackball, by which the user can provide input to the computer. Other kinds of devices can be used to provide interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's client device in response to requests received from the web browser.

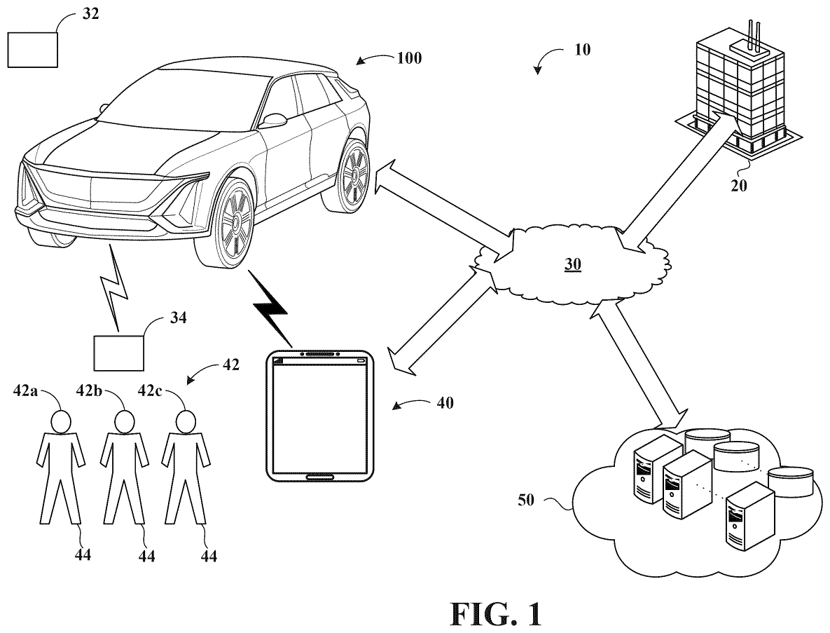

Referring to FIG. 1, an example vehicle operating environment 10 is provided for illustration of the principles of the present disclosure. The vehicle operating environment 10 includes a vehicle 100 and a vehicle service center 20. For the sake of illustration, the vehicle operating environment 10 is shown as including a single vehicle service center 20. However, in other examples, the vehicle operating environment 10 may include a plurality of vehicle service centers 20 in communication over a network 30 (e.g., the Internet, cellular networks). The vehicle operating environment 10 also includes one or more detection devices 32, such as one or more cameras or one or more sensors. In at least one example, the one or more detection devices 32 can be coupled to or arranged within a vehicle-friendly infrastructure (e.g., a parking garage). Additionally, the vehicle operating environment can include a key fob 34 in communication with the vehicle 100 and/or a mobile device 40 in communication with the vehicle 100 and/or the network 30. The key fob 34 and/or the mobile device 40 can be configured so that a user (e.g., driver or another passenger) can gain access to the vehicle 100, for example.

With continued reference to FIG. 1, one or more individuals 42 can be included in the vehicle operating environment 10. In the present illustrative configuration, the one or more individuals can include a driver 42a, a passenger 42b of the vehicle 100, and a pedestrian 42c. The one or more individuals 42 each includes a gait pattern 44. For purposes of the present application, the gait pattern 44 can refer to a pattern of walking (i.e., stride length, cadence, speed, hip/knee/foot angles, etc.). Certain injuries, medical conditions, and other factors (i.e., fatigue, stress, etc.) can affect the gait pattern 44 of the one or more individuals 42. As will be discussed in more detail below, collecting and assessing the gait pattern 44 of the one or more individuals 42 approaching or surrounding the vehicle 100 can be desirable so that one or more aspects of the vehicle 100 can be adjusted to accommodate one or more of the gait patterns 44, for example.

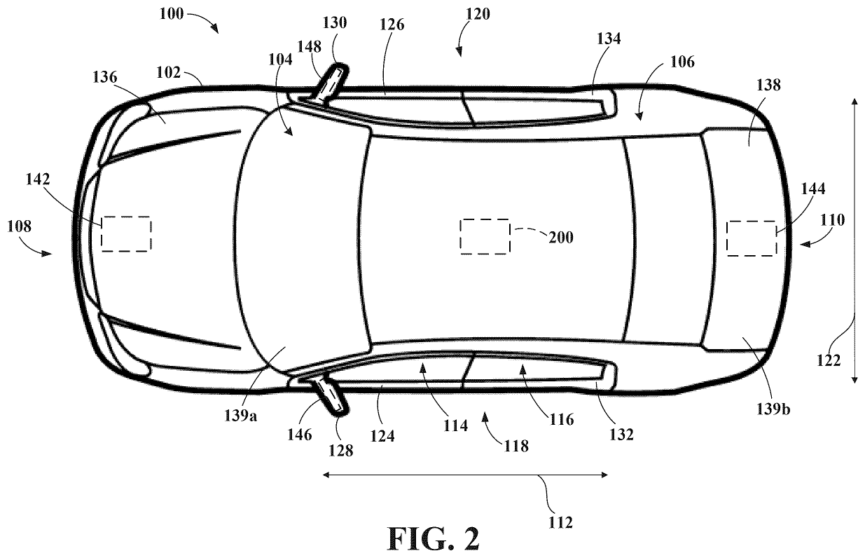

With reference to FIG. 2, the vehicle 100 can have a vehicle body 102, which has a vehicle interior 104 and a vehicle exterior 106. The vehicle body 102 has a first or front end 108 spaced fore-aft from a second or rear end 110 with respect to a longitudinal axis 112. The vehicle 100 has a first or front passenger compartment 114 near the front end 108 and a second or rear passenger compartment 116 spaced fore-aft from the front passenger compartment 114 toward the rear end 110. In this example, the front passenger compartment 114 and the rear passenger compartment 116 each have a first or driver side 118 and a second or co-pilot side 120 spaced cross-car from the driver side 118 with respect to a lateral axis 122. The lateral axis 122 is perpendicular to the longitudinal axis 112. The driver and co-pilot sides 118, 120 may be on different sides (i.e., left or right) depending on the region of the world for which the vehicle 100 is manufactured. For the purposes of the present disclosure, the driver side 118 is on the left side and the co-pilot side 120 is on the right side of the vehicle 100 with respect to the lateral axis 122. Note, the present disclosure equally applies to vehicles where the driver side is on the right side of the vehicle and the co-pilot side is on the left side of the vehicle.

With continued reference to FIG. 2, one or more closures (e.g., doors, tailgate, etc.) may be coupled to the vehicle body 102 of the vehicle 100. For instance, a front left passenger compartment door 124 and a front right passenger compartment door 126 may be coupled to the vehicle body 102 to enclose the front passenger compartment 114. The front left passenger compartment door 124 and the front right passenger compartment door 126 can each include side view mirrors 128, 130. The side view mirrors 128, 130 can include a motor or another mechanism that is configured to be automatically adjusted (i.e., position, angle, etc.). A rear left passenger compartment door 132 and a rear right passenger compartment door 134 can be coupled to the vehicle body 102 and enclose the rear passenger compartment 116. The vehicle body 102 can also include a front hood 136 arranged at the front end 108 and a liftgate 138 (i.e., a rear hood) arranged at the rear end 110. The front hood 136 and the liftgate 138 can each include a motor or another mechanism that is configured to receive and process instructions to open and/or close the front hood 136 and the liftgate 138. Additionally, the vehicle 100 can include a front windshield 139a arranged adjacent to the front hood 136 at the front end 108 and extends cross-car between the driver side 118 and the passenger side 120. Similarly, the vehicle 100 can include a rear windshield 139b arranged adjacent the liftgate 138 at the rear end 110 and extends cross-car between the driver side 118 and the passenger side 120.

With continued reference to FIG. 2, the vehicle 100 can include a sensor system 140 that includes one or more sensors arranged within the vehicle interior 104 and/or the vehicle exterior 100. For instance, the one or more sensors can include a first sensor 142 coupled to the vehicle body 102 toward the front end 108, a second sensor 144 coupled to the vehicle body 102 toward the rear end 110, a third sensor 146 coupled to the vehicle body 102 on the driver side 118, and a fourth sensor 148 coupled to the vehicle body 102 on the passenger side 120. In the present illustrative configuration, the first sensor 142 can be coupled to or arranged adjacent to the front hood 136, the second sensor 144 can be coupled to or arranged adjacent to the liftgate 138, the third sensor 146 can be coupled to or arranged adjacent to the side view mirror 128 of the front left passenger compartment door 124, and the fourth sensor 148 can be coupled to or arranged adjacent to the side view mirror 130 of the front right passenger compartment door 126. The first, second, third, and fourth sensors 142, 144, 146, 148 can include one or more cameras, radars, wheel speed sensors, impact sensors, etc. In general, the one or more sensors can be configured to gather and/or capture information (i.e., data) concerning one or more aspects surrounding the vehicle 100 during travel and/or while at rest. More particularly, the one or more sensors can be configured to capture information concerning the gait pattern 44 of the one or more individuals 42 approaching or surrounding the vehicle 100.

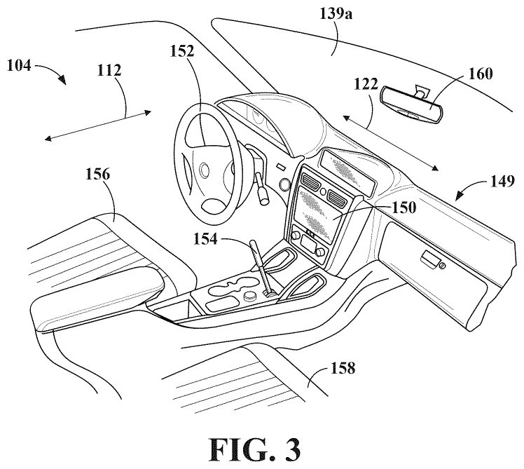

With reference to FIG. 3, an example configuration of the vehicle interior 104 of the vehicle 100 is provided. An instrument panel 149 is arranged in the vehicle interior 104 and extends with respect to the lateral axis 122. An infotainment system or display 150 can be coupled to or arranged on or in the instrument panel 149. The infotainment system 150 can be configured to act as a user interface for controlling settings of the vehicle 100, such as heating, ventilation, and air conditioning (HVAC), for example. The infotainment system 150 can be configured to receive one or more inputs and adjust settings of the vehicle 100 accordingly. A steering wheel 152 can be coupled to the instrument panel 149 and extend fore-aft with respect to the longitudinal axis 112. The steering wheel 152 can include a motor (not shown) or another mechanism that is configured to be automatically adjusted (e.g., position, height, etc.). A shifter 154 may be arranged cross-car between a front left seat 156 and a front right seat 158 so that an operator of the vehicle 100 can change a gear status (i.e., park, reverse, neutral, drive, low) of the vehicle 100. The front left seat 156 can be arranged fore-aft of the steering wheel 152 and can include a motor or another mechanism that is configured to be automatically adjusted (e.g., seat height, position, recline, etc.). Similarly, the front right seat 158 can include a motor or another mechanism that is configured to be automatically adjusted (e.g., seat height, position, recline, etc.). A rearview mirror 160 can be coupled to an interior portion of the front windshield 139a and arranged cross-car with respect to the lateral axis 122. The rearview mirror 160 can include a motor or another mechanism that is configured to be automatically adjusted (e.g., position, angle, etc.).

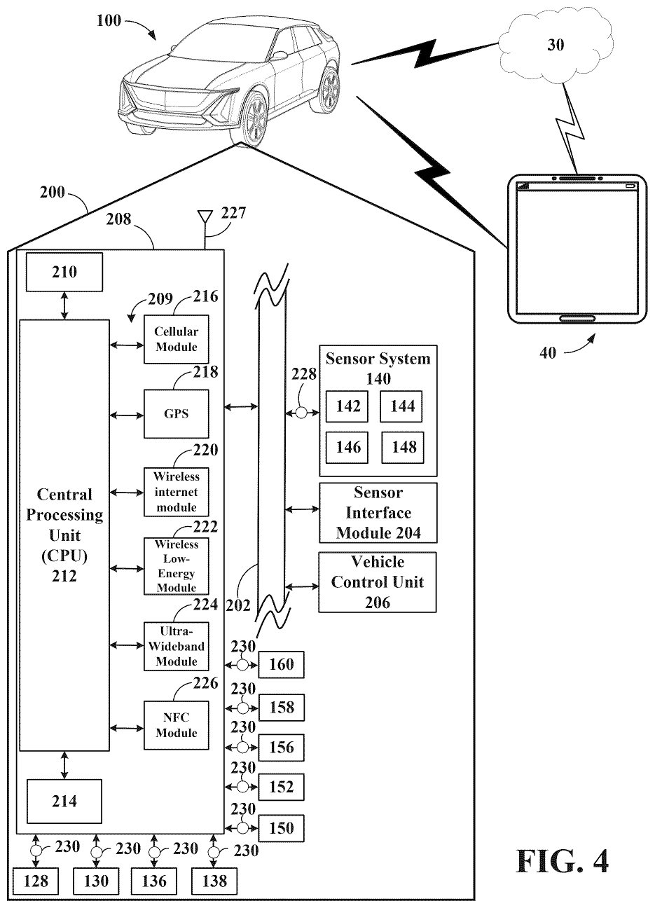

With reference to FIG. 4, the vehicle 100 may be equipped with a vehicle management system 200 (e.g., a telematics unit) that comprises a network connection interface 202. In the present example, the network connection interface 202 is communicatively coupled to the vehicle management system 200. Some examples of the network connection interface 202 may include a twisted pair/fiber optic Ethernet switch, an internal/external parallel communication bus, a local area network (LAN) interface, a controller area network (CAN), a media-oriented system transfer (MOST), a local interconnection network (LIN) interface, and the like. Other communication interfaces may also include those that conform with ISO, SAE, and IEEE standards and specifications. The network connection interface 202 enables components of the vehicle management system 200 to send and receive signals with each other and with various systems and subsystems both within or “resident” to the vehicle body 102 and outside or “remote” from the vehicle body 102. This allows the vehicle 100 to perform various vehicle functions, such as communicating with the mobile device 40 via peer-to-peer communication or via the network 30 (i.e., through cellular or wireless internet). The vehicle management system 200 can receive and/or transmit data to/from one or more electronic control units (ECUs) such as a sensor interface module 204 and a vehicle control unit (VCU) 206, for example. The vehicle management system 200 may also communicate with additional ECUs such as an engine control module (ECM), a powertrain control module (PCM), a transmission control module, a brake system control module (BSCM), a climate control module (CCM), etc.

With continued reference to FIG. 3, the vehicle management system 200 can include a communication system 208 (e.g., an onboard computing device) that provides several functions, both individually and through its communication with other networked devices. For instance, the communication system 208 can wirelessly communicate (e.g., via cell towers, base stations, and/or mobile switching centers (MSCs), etc.) with a remotely located or off-board cloud computing system 50 (FIG. 1) via the network 30 or with the mobile device 40. The communication system 208 is generally composed of data processing hardware 210, each of which may be embodied as a discrete microprocessor, an application specific integrated circuit (ASIC), or a dedicated control module. The vehicle 100 may offer centralized vehicle control via a central processing unit (CPU) 212 that is operatively coupled to memory hardware 214, each of which may take on the form of a CD-ROM, magnetic disk, IC device, semiconductor memory (e.g., various types of RAM or ROM), etc. The vehicle 100 can include wireless communication technology 209 comprising long-range vehicle communication capabilities and close-range vehicle communication capabilities. The long-range vehicle communication capabilities with remote, off-board networked devices may be provided via one or more or all of a cellular chipset or module 216, a navigation and location chipset or module 218 (e.g., global positioning system (GPS)), or a wireless internet (Wi-Fi®) chipset or module 220. The close-range wireless connectivity may be provided via a close-range wireless communication device such as a wireless low-energy chipset or module 222 (e.g., a Bluetooth® Low Energy (BLE)), an ultra-wideband (UWB) chipset or module 224, and a near field communications (NFC) chipset or module 226, and/or a dual antenna 227.

The CPU 212 may receive data from the one or more sensors of the sensor system 140. Additionally, the CPU can receive data from any of the one or more sensors that are configured for cellular communication, wireless internet communication, low-energy wireless communication, ultra-wideband communication, and/or near-field communication, or for other communication with the network 30 and/or the mobile device 40. The vehicle 100 and, more particularly, the sensor system 140 may be equipped with one or more sensors for the wireless communication technology 209. In other words, at least some of the one or more sensors of the sensor system 140 can be configured to receive wireless communication signals of cellular communication, wireless internet communication, low-energy wireless communication, ultra-wideband communication, and/or near-field communication, for example.

The detection device 32 can be configured to communicate with the wireless communication technology 209 directly or via the network 30. According to at least one aspect, the detection device 32 can be configured to gather data 228 concerning the gait pattern 44 of the one or more individuals 42 and communicate it to the vehicle 100 or the network 30 for storage or evaluation.

The key fob 34 and the mobile device 40 (e.g., smartphone) can be configured to communicate with the wireless communication technology 209 directly or via the network 30. According to one aspect, the vehicle 100 and, more particularly, the vehicle management system 200, can be configured to wake up the vehicle 100 when the key fob 34 and/or the mobile device 40 is detected within a proximity of the vehicle 100. As will be discussed in more detail below, once the key fob 34 and/or the mobile device 40 is detected, the sensor system 140 can begin collecting data 228 concerning the one or more individuals 42 within the proximity of the vehicle 100, for example. In at least one configuration, the sensor system 140 can be configured to continuously collect data 228, even during a low-power mode, for example.

With continued reference to FIG. 4, the motor or other mechanisms coupled to each of the side view mirrors 128, 130, the front hood 136, and the liftgate 138 can be communicatively coupled to the communication system 208 and configured to receive instructions 230 from the vehicle control unit 206 or another module within the vehicle 100. Similarly, the infotainment system 150 can be communicatively coupled to the communication system 208 and configured to receive instructions 230 from the vehicle control unit 206 or another module within the vehicle 100. The motor or other mechanisms coupled to each of the steering wheel 152, the front left seat 156, the front right seat 158, and the rearview mirror 160 can be communicatively coupled to the communication system 208 and configured to receive instructions 230 from the vehicle control unit 206 or another module within the vehicle 100. Note, other aspects of the vehicle 100 may also be communicatively coupled to the communication system 208 such as a vehicle suspension, a vehicle powertrain, a safety restraint system (e.g., seat belts, airbags, etc.). These systems can be configured to receive instructions 230 from the vehicle control unit 206 or another module within the vehicle 100.

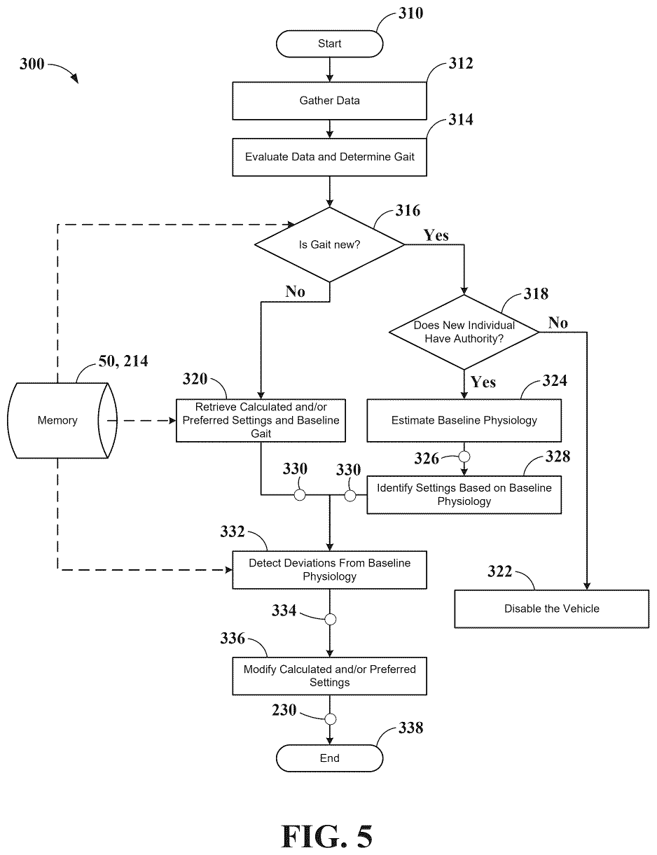

According to the principles of the present disclosure, with reference to FIG. 5, a method 300 is provided and, in general, evaluates the gait patterns 44 of the one or more individuals 42 within the vehicle operating environment 10 and adjusts one or more aspects of the vehicle 100 accordingly. Accommodating the gait patterns 44 of the one or more individuals 42 can be desirable for enhancing and/or improving a driving experience, for example. The method 300 is initiated at 310. In practical terms, the method 300 can be initiated when at least one of the one or more sensors collects data that indicates the one or more individuals 42 are approaching the vehicle 100. For instance, if the one or more individuals 42 unlock the vehicle 100 using the key fob 34 or the mobile device 40, for example, the method 300 will be initiated. In general, the method 300 can be initiated in any instance where the one or more individuals 42 can be evaluated by the one or more sensors of the sensor system 140 or the one or more detection devices 32 in communication with the vehicle 100.

At 312, the one or more individuals 42 are evaluated by the one or more sensors (e.g., the first, second, third, and/or fourth sensors 142, 144, 146, 148) and the data 228 can be stored locally in the memory hardware 214 and/or remotely in the off-board cloud computing system 50.

At 314, the data 228 is evaluated and/or analyzed to determine the gait patterns 44 of the one or more individuals 42. The data 228 can be processed using one or more machine learning (ML) algorithms, such as convolutional long short-term memory (ConvLSTM), vision transformers, graph neural networks, Bayesian networks, deep Gaussian processes, multimodal large language models (LLM), vision language models (VLM), as well as other ML algorithms utilized in the automotive industry. Utilizing one or more of the ML algorithms can be desirable to analyze the data to establish the gait patterns 44 for each of the one or more individuals 42.

At 316, the gait patterns 44 of the one or more individuals 42 can be evaluated (i.e., compared) against the data 228 stored in the memory hardware 214 and/or in the off-board cloud computing system 50 to identify or determine whether one or more of the one or more individuals 42 have already been evaluated. If the gait pattern 44 is new (i.e., has not been previously identified) then the method 300 proceeds to 318. If the gait pattern 44 is not new (i.e., has been previously identified) then the method proceeds to 320.

At 318, new gait patterns 44 can require vehicle owner authorization. For instance, the gait patterns 44 can include one concerning the pedestrian 42c who does not own the vehicle 100 or have authority to enter the vehicle 100. An owner or an authorized user of the vehicle 100 can be notified via an application, text, and/or call on the mobile device 40 and choose to either approve or disprove a particular individual. If the owner or the authorized user does not approve one of the one or more individuals 42 (e.g., the pedestrian 42c) then the vehicle 100 can be disabled at 322. If the owner or authorized user approves the one or more individuals 42 whose gait 44 has not been previously identified, then the method 300 proceeds to 324. In at least one configuration, facial recognition can also be used to determine whether one of the one or more individuals 42 is an authorized user of the vehicle 100.

At 324, a baseline physiology 326 of the new individual of the one or more individuals 42 is estimated. In other words, the height, body mass index, age, sex, etc. of the new individual of the one or more individuals 42 is estimated. According to at least one aspect, the one or more ML algorithms introduced above can be used to help estimate and/or establish the baseline physiology 326 of the new individual of the one or more individuals 42.

At 328, the baseline physiology 326 can be used to establish one or more calculated settings 330 and/or features of the vehicle 100. For instance, if the new individual is approaching the front left passenger compartment door 124 and is going to be driving the vehicle 100, the position of the front left seat 156, steering wheel 152, rearview mirror 160, side view mirrors 128, 130, etc., can be determined. Other aspects of the vehicle 100 can be determined as well, such as a driving mode (e.g., fully automated, partially automated, manual, etc.). The calculated settings 330 can be stored locally in the memory hardware 214 and/or remotely in the off-board cloud computing system 50. Additionally, if the new individual of the one or more individuals 42 enters the vehicle 100 for the first time and changes various settings from the calculated settings 330, the personal preferences can be stored in addition to or in place of the calculated settings 330.

At 320, when one of the or more individuals 42 have been previously identified then their calculated or preferred settings 330 can be retrieved from the memory hardware 214 or from the off-board cloud computing system 50.

At 332, the data 228 can be evaluated against the baseline physiology 326 and calculated or preferred settings 330 to determine if there are any deviations 334. One or more of the deviations 334 can exist due to carrying an object (e.g., baby, shopping bag, etc.) and/or due to fatigue, stress, agitation, disability, for example. Other factors not provided here can result in a deviation from the gait pattern 44 of the one or more individuals 42 as well. According to at least one aspect, the one or more ML algorithms introduced above can be used to help determine whether any of the deviations exist.

At 336, the calculated or preferred settings 330 can be modified to accommodate the deviations 334 so that the instructions 230 can be determined. For instance, if the gait pattern 44 of one of the one or more individuals 42 is affected due to carrying grocery bags, one of the settings 330 may be modified so that the instructions 230 will open the liftgate 138 (i.e., rear hood) automatically, for example.

At 338, the instructions 230 can be communicated to one or more aspects of the vehicle 100 via the communication system 208 so that one or more settings of the vehicle 100 are adjusted to enhance or improve the experience of the one or more individuals (e.g., the driver 42a and the passenger 42b) and then the method 300 ends.

A number of implementations have been described. Nevertheless, it will be understood that various modifications may be made without departing from the spirit and scope of the disclosure. Accordingly, other implementations are within the scope of the following claims.

The foregoing description has been provided for purposes of illustration and description. It is not intended to be exhaustive or to limit the disclosure. Individual elements or features of a particular configuration are generally not limited to that particular configuration, but, where applicable, are interchangeable and can be used in a selected configuration, even if not specifically shown or described. The same may also be varied in many ways. Such variations are not to be regarded as a departure from the disclosure, and all such modifications are intended to be included within the scope of the disclosure.

What is claimed is:

1. A computer-implemented method that, when executed by data processing hardware, causes the data processing hardware to perform operations comprising:
detecting one or more individuals within a proximity of a vehicle;
gathering data concerning the one or more individuals;
calculating a baseline gait for each of the one or more individuals;
determining a physiology for each of the one or more individuals based on the data and the baseline gait; and
calculating one or more vehicle settings based on the physiology and the baseline gait for each of the one or more individuals.

2. The method of claim 1, further including:
determining whether the data indicates one or more deviations from the baseline gait; and
modifying the vehicle settings based on the one or more deviations.

3. The method of claim 1, wherein detecting the one or more individuals within the proximity of the vehicle further includes detecting a key fob or a mobile device.

4. The method of claim 1, wherein gathering the data concerning the one or more individuals further includes capturing the data using one or more sensors coupled to the vehicle.

5. The method of claim 1, further including identifying the one or more individuals using facial recognition.

6. The method of claim 1, wherein calculating the one or more vehicle settings based on the physiology and the baseline gait for each of the one or more individuals further includes calculating a position of one or more seats arranged within the vehicle.

7. The method of claim 1, wherein calculating the one or more vehicle settings based on the physiology and the baseline gait for each of the one or more individuals further includes calculating a position of a steering wheel arranged within the vehicle.

8. The method of claim 1, wherein calculating the one or more vehicle settings based on the physiology and the baseline gait for each of the one or more individuals further includes opening a liftgate of the vehicle.

9. The method of claim 1, wherein calculating the one or more vehicle settings based on the physiology and the baseline gait for each of the one or more individuals further includes calculating a position of one or more sideview mirrors.

10. The method of claim 1, wherein calculating the one or more vehicle settings based on the physiology and the baseline gait for each of the one or more individuals further includes calculating a position of a rearview mirror.

11. A system comprising:
data processing hardware; and
memory hardware in communication with the data processing hardware, the memory hardware storing instructions that, when executed on the data processing hardware, cause the data processing hardware to perform operations comprising:
detecting one or more individuals within a proximity of a vehicle;
gathering data concerning the one or more individuals;
calculating a baseline gait for each of the one or more individuals;
determining a physiology for each of the one or more individuals based on the data and the baseline gait; and
calculating one or more vehicle settings based on the physiology and the baseline gait for each of the one or more individuals.

12. The system of claim 11, further including:
determining whether the data indicates one or more deviations from the baseline gait; and
modifying the vehicle settings based on the one or more deviations.

13. The system of claim 11, wherein detecting the one or more individuals within the proximity of the vehicle further includes detecting a key fob or a mobile device.

14. The system of claim 11, wherein gathering the data concerning the one or more individuals further includes capturing the data using one or more sensors coupled to the vehicle.

15. The system of claim 11, further including identifying the one or more individuals using facial recognition.

16. A vehicle management system comprising:
a sensor system including one or more sensors coupled to a vehicle;
data processing hardware; and
memory hardware in communication with the data processing hardware, the memory hardware storing instructions that, when executed on the data processing hardware, cause the data processing hardware to perform operations comprising:
detecting one or more individuals within a proximity of the vehicle;
gathering data concerning the one or more individuals;
calculating a baseline gait for each of the one or more individuals;
determining a physiology for each of the one or more individuals based on the data and the baseline gait; and
calculating one or more vehicle settings based on the physiology and the baseline gait for each of the one or more individuals.

17. The vehicle management system of claim 16, further including:
determining whether the data indicates one or more deviations from the baseline gait; and
modifying the vehicle settings based on the one or more deviations.

18. The vehicle management system of claim 16, wherein detecting the one or more individuals within the proximity of the vehicle further includes detecting a key fob or a mobile device.

19. The vehicle management system of claim 16, wherein gathering the data concerning the one or more individuals further includes capturing the data using the one or more sensors.

20. The vehicle management system of claim 16, further including identifying the one or more individuals using facial recognition.
